(19) United States
(12) Patent Application Publication
(10) Pub. No.: US 2020/0147217 A1
(43) Pub. Date: May 14, 2020

(54) FUCOIDAN-BASED THERAGNOSTIC COMPOSITION

(71) Applicant: National Cancer Center, Goyang-si (KR)

(72) Inventors: Yong Doo CHOI, Goyang-si (KR); Mi Hyeon Cho, Goyang-si (KR); Yan Li, Seoul (KR)

(73) Assignee: National Cancer Center, Goyang-si (KR)

(21) Appl. No.: 16/677,104

(22) Filed: Nov. 7, 2019

(30) Foreign Application Priority Data
Nov. 8, 2018 (KR) KR10-2018-0136351

(51) Int. Cl. A61K 41/00; A61K 47/61; A61K 49/00; A61K 47/54

(52) U.S. Cl. CPC A61K 41/0071; A61K 47/549; A61K 49/0054; A61K 47/61

(57) ABSTRACT

The present invention relates to a fucoidan-based theragnostic composition, and more particularly, to preparation and use of a theragnostic composition which uses a conjugate of a fluorescent dye or a photosensitizer and fucoidan, thereby not only allowing for fluorescence imaging diagnosis of lesions for cancer or vascular diseases but also allowing a therapeutic effect thereon to be obtained at the same time. The conjugate obtained by covalent bonding of a fluorescent dye or a photosensitizer and fucoidan, according to the present invention, is not only useful for fluorescence imaging diagnosis of tumor tissues and ophthalmic vascular diseases, but also may exhibit a therapeutic effect on cancer cells and coronary artery smooth muscle cells, and a neovascularization inhibitory effect in ophthalmic diseases. In addition, in a case where photodynamic therapy is further implemented on the conjugate according to the present invention, cancer and vascular diseases may be effectively treated with low adverse effects.

### BACKGROUND OF THE INVENTION

### 1. Field of the Invention

The present invention relates to a fucoidan-based theragnostic composition, and more particularly, to preparation and use of a theragnostic composition which uses a conjugate obtained by covalent bonding of a fluorescent dye or a photosensitizer and fucoidan, thereby not only allowing for fluorescence imaging diagnosis of lesions for cancer or vascular diseases, but also allowing a therapeutic effect thereon to be obtained at the same time.

### 2. Description of the Related Art

Diagnosis and therapy are two main categories in clinical application for diseases, and a concept of theragnosis has recently been introduced, which is a technique for simultaneously performing diagnosis and therapy by utilizing a therapeutic agent having an imaging function.

A photosensitizer used in photodynamic therapy (PDT) has a characteristic of absorbing light of a certain wavelength and generating a fluorescence signal. The photosensitizer is used for fluorescence imaging diagnosis of disease lesions using such a characteristic or has an advantage of selectively killing only target cells using singlet oxygen or free radical, which is a reactive oxygen species that is generated only at the site irradiated with light while minimizing adverse effects seen in anticancer drugs or the like. Research on photodynamic therapy has been actively conducted since the early 20th century. In the present, the photodynamic therapy has been used for diagnosis and therapy of cancer, therapy of ophthalmic diseases, therapy of vascular diseases such as arteriosclerosis, therapy of acne and dental diseases, and used to increase immunity to autologous bone marrow transplantation, antibiotics, therapy of AIDS, skin transplantation surgery or therapy of arthritis or the like, so that its application range is gradually expanding. In recent years, combination of photodynamic therapy with immunotherapy opens a possibility of treating even metastatic cancer which is located at the site not irradiated with light. With development of technology, use of photosensitizers as therapeutic agents for sonodynamic therapy in recent years has also made it possible to treat tumors located deep in the human body, which were difficult to treat with conventional photodynamic therapy. In addition, selective fluorescence imaging diagnostic techniques for various disease lesions have been developed using near-infrared fluorescent dyes and are clinically applied. Photosensitizers also generate strong fluorescence signals in a case of being irradiated with light of a certain wavelength. Therefore, using this characteristic of photosensitizers, efforts have also been actively made to apply photosensitizers to fluorescence imaging diagnosis of disease lesions such as cancer.

Conventionally used photosensitizers for photodynamic diagnosis or therapy are hydrophobic, which causes nonspecific accumulation thereof in normal tissues including skin, in addition to cancer tissues, after being administered to patients by intravenous injection (see Korean Laid-open Patent Publication No. 10-2008-0095182). This lowers a target-to-background ratio, which not only makes it difficult to achieve imaging diagnosis of a tumor site, but also causes risks of damaging peripheral important normal tissues during photodynamic therapy. In addition, when a patient who has undergone photodynamic therapy is exposed to bright light such as sunlight, production of reactive oxygen is activated from photosensitizers that have been accumulated in the skin, which may cause skin photosensitivity that is an adverse effect. For this reason, after photodynamic therapy, patients are advised to stay in the dark room for at least six weeks until the photosensitizers which have been accumulated in normal tissues such as skin disappear, which causes inconvenience to the patients. Attempts have been made to address skin photosensitivity problems by increasing hydrophilicity of photosensitizers. However, in this case, large amounts of intravenously administered photosensitizers are rapidly excreted through urine, and thus there is a disadvantage that a high dose of photosensitizers must be administered to cause a therapeutically sufficient amount of photosensitizers to be accumulated in tumor tissues.

In a case a photosensitizer is conjugated, via a covalent bond, to a hydrophilic polymer such as chitosan, glycol chitosan, poly(ethylene glycol), poly-L-lysine, and carboxymethyl dextran, it is possible to obtain an effect of stably dispersing the photosensitizer in an aqueous solution while increasing accumulation efficiency thereof against tumor (see Korean Laid-open Patent Publication No. 10-2017-0048202). However, while this hydrophilic polymer-photosensitizer conjugate helps improve hydrophilicity of the photosensitizer, such a conjugate has no target specificity for cancer cells or cells associated with other diseases. Therefore, in order for the conjugate to have specificity for target cells, a target ligand such as folic acid, antibody, or aptamer had to be further conjugated to the polymer. In this case, steps and costs for manufacturing are increased, and mass production of the conjugate becomes difficult. In addition, even though the hydrophilic polymer occupies 70% or more of the mass of the hydrophilic polymer-photosensitizer conjugate, a therapeutic effect can be obtained only by the photosensitizer, and the hydrophilic polymer itself has no therapeutic effect on cancer cells or the like. Therefore, such a conjugate has a big disadvantage that a low therapeutic effect may be obtained relative to its mass, and no photodynamic therapeutic effect is obtained in a case where light does not reach the site where the polymer-photosensitizer conjugate is delivered.

In addition, attempts are made to achieve imaging diagnosis of lesions such as cancer using a conjugate (that is, a ligand-fluorescent dye-hydrophilic polymer conjugate) of a fluorescent dye, to which a ligand capable of targeting a specific cell is bound, and a hydrophilic polymer. In this case, only an imaging diagnostic function for the target site can be obtained; and in order to obtain a therapeutic effect, a complicated process of additional conjugation or loading of a drug onto the polymer is required.

### SUMMARY OF THE INVENTION

In order to overcome the above problems, the present inventors have prepared a conjugate in which fucoidan is covalently bonded to a photosensitizer or a fluorescent dye, and have identified that the conjugate not only allows for simultaneous achievement of fluorescence imaging and photodynamic therapy, but also allows even a therapeutic effect and a target specificity effect of fucoidan to be obtained at the same time, so that an improved therapeutic effect that could not be expected previously can be obtained, thereby completing the present invention.

Accordingly, an object of the present invention is to provide a conjugate in which fucoidan and a fluorescent dye, or fucoidan and a photosensitizer are conjugated to each other via a covalent bond.

Another object of the present invention is to provide a composition for fluorescence imaging diagnosis and a composition for photodynamic therapy, using the conjugate, which not only enable real-time fluorescence imaging diagnosis of a site, with respect to cancer and vascular-related diseases such as atherosclerotic plaques, or ophthalmic diseases such as senile macular degeneration and glaucoma, but also can have a therapeutic effect on such diseases.

Unless defined otherwise, all technical and scientific terms used herein have the same meaning as commonly understood by those skilled in the art to which the present invention belongs. In general, the nomenclature used herein is well known and commonly used in the art.

In order to solve the technical problems as described above, the present invention provides a conjugate in which fucoidan is covalently bonded to a photosensitizer or a fluorescent dye, and a composition for fluorescence imaging diagnosis or a composition for photodynamic therapy, comprising the same.

Hereinafter, the present invention will be described in more detail.

In an aspect, the present invention relates to a conjugate in which a photosensitizer or a fluorescent dye is covalently bonded to fucoidan. Here, the conjugate includes the same meaning as combination or assembly.

In an aspect, the present invention relates to a fluorescent dye-fucoidan conjugate in which fucoidan and a fluorescent dye are covalently bonded to each other.

The present invention may be characterized in that a carboxyl group of the fucoidan and an amine group of the fluorescent dye are covalently bonded to each other using a coupling agent.

In the present invention, the fluorescent dye may be a fluorescent dye selected from the group consisting of cyanine, rhodamine, coumarin, EvoBlue, oxazine, BODIPY, carbopyronine, naphthalene, biphenyl, anthracenes, phenanthrene, pyrene, carbazole, or derivatives based on the above-mentioned dyes.

In an embodiment of the present invention, the fluorescent dye may be selected from the group consisting of Fluorescein, CR110: Carboxyrhodamine 110: Rhodamine Green (trade name), TAMRA: carboxytetramethylrhodamine: TMR, Carboxyrhodamine 6G: CR6G, BODIPY FL (trade name): 4,4-difluoro-5,7-dimethyl-4-bora-3a,4a-diaza-s-indacene-3 -propionic acid, BODIPY 493/503 (trade name): 4,4-difluoro-1,3,5,7-tetramethyl-4-bora-3a,4a-diaza-s-indacene-8-propionic acid, BODIPY R6G (trade name): 4,4-difluoro-5-(4-phenyl-1,3-butadienyl)-4-bora-3a,4a-diaza-s-indacene-3-propionic acid, BODIPY 558/568 (trade name): 4,4-difluoro-5-(2-thienyl)-4-bora-3a,4a-diaza-s-indacene-3-propionic acid, BODIPY 564/570 (trade name): 4,4-difluoro-5-styryl-4-bora-3a,4a-diaza-s-indacene-3-propionic acid, BODIPY 576/589 (trade name): 4,4-difluoro-5-(2-pyrolyl)-4-bora-3a,4a- diaza-s-indacene-3-propionic acid, BODIPY 581/591 (trade name): 4,4- difluoro-5 -(4-phenyl-1,3-butadienyl)-4-bora-3a,4a-diaza-s-indacene-3-propionic acid, EvoBlue10 (trade name), EvoBlue30 (trade name), MR121, ATTO 655 (trade name), ATTO 680 (trade name), ATTO 700 (trade name), ATTO MB2 (trade name), Alexa Fluor 350 (trade name), Alexa Fluor 405 (trade name), Alexa Fluor 430 (trade name), Alexa Fluor 488 (trade name), Alexa Fluor 532 (trade name), Alexa Fluor 546 (trade name), Alexa Fluor 555 (trade name), Alexa Fluor 568 (trade name), Alexa Fluor 594 (trade name), Alexa Fluor 633 (trade name), Alexa Fluor 680 (trade name), Alexa Fluor 700 (trade name), Alexa Fluor 750 (trade name), Alexa Fluor 790 (trade name), Flamma 496 (trade name), Flamma 507 (trade name), Flamma 530 (trade name), Flamma 552 (trade name), Flamma 560 (trade name), Flamma 575 (trade name), Flamma 581 (trade name), Flamma 648 (trade name), Flamma 675 (trade name), Flamma 749 (trade name), Flamma 774 (trade name), Flamma 775 (trade name), Rhodamine Red-X (trade name), Texas Red-X (trade name), 5(6)-TAMRA-X (trade name), 5TAMRA (trade name), Cy5™, Cy5.5™, Cy7™ or Licor NIR™, IRDye38™, IRDye78™, IRDye80™, LaJolla Blue™, Licor NIR™, Indocyanine green (ICG), and ZW800-1C.

In the present invention, a binding ratio of fucoidan to fluorescent dye is preferably 1:2 to 1:4; and in a case where more fluorescent dye is intended to be bound thereto, it is preferable to cause the fucoidan and the fluorescent dye to be bound to each other via a linker that may be decomposed in a target cell.

The fluorescent dye-fucoidan conjugate according to the present invention may be prepared by a method which comprises a step (first step) of dissolving fucoidan in a buffer solution; a step (second step) of adding a coupling agent to the dissolved product of the first step and performing stirring; a step (third step) of removing the reaction mixture of the second step, adding a near-infrared fluorescent dye thereto, and performing stirring; and a step (fourth step) of subjecting the reaction mixture of the third step to dialysis against distilled water, and then performing freeze-drying, to obtain a fluorescent dye-fucoidan conjugate in which the near-infrared fluorescent dye is covalently bonded to the fucoidan. However, the present invention is not limited to the method.

Since the fluorescent dye-fucoidan conjugate according to the present invention can retain high binding specificity for P-selectin and vascular endothelial growth factor even after formation of the conjugate, such a conjugate allows for fluorescence imaging diagnosis of neovascularization sites in cancer cells, atherosclerotic plaques, and ophthalmic diseases, and of platelet-rich thrombi.

Therefore, the present invention relates to a composition for fluorescence imaging diagnosis, comprising the fluorescent dye-fucoidan conjugate.

In an aspect, the present invention relates to a photosensitizer-fucoidan conjugate in which fucoidan and a photosensitizer are covalently bonded to each other.

In the present invention, the photosensitizer and the fucoidan are bonded to each other, using a linker containing a disulfide or diselenide bond which acts on a carboxyl group of the fucoidan.

Such a photosensitizer may be selected from, but is not limited to, the group consisting of: a porphyrin-based compound selected from the group consisting of hematoporphyrins, porphycenes, pheophorbides, purpurins, chlorins, protoporphyrins, and phthalocyanines; and a non-porphyrin-based compound selected from the group consisting of hypericin, rhodamine, ATTO, rose Bengal, psoralen, phenothiazinium-based dyes, and merocyanine.

The photosensitizer-fucoidan conjugate according to the present invention may be prepared by a method which comprises a step (first step) of dissolving fucoidan in a buffer solution; a step (second step) of adding a coupling agent to the dissolved product of the first step and performing stirring; a step (third step) of adding, to the reaction mixture of the second step, a linker (linker) containing a disulfide or diselenide bond, and performing stirring; a step (fourth step) of subjecting the reaction mixture of the third step to dialysis against distilled water, and then performing freeze-drying, to obtain a fucoidan derivative having an amine group; a step (fifth step) of dissolving a photosensitizer in an organic solvent, adding a coupling agent thereto, and performing stirring; a step (sixth step) of mixing the fucoidan derivative having an amine group with the reaction solution of the fifth step so that reaction is allowed to proceed; and a step (seventh step) of subjecting the reaction mixture of the sixth step to dialysis against phosphate buffer and distilled water, and then performing freeze-drying, to obtain a photosensitizer-fucoidan conjugate in which the photosensitizer and the fucoidan are covalently bonded to each other via the linker. However, the present invention is not limited to the method.

In the photosensitizer-fucoidan conjugate according to the present invention, the fucoidan polymer itself exhibits direct cytotoxicity against cancer cells or smooth muscle cells, or an effect of inhibiting cancer growth or inhibiting neovascularization in ophthalmic diseases is further obtained due to binding of the fucoidan with vascular endothelial growth factor (VEGF), so that a therapeutic effect caused by the fucoidan itself and a photodynamic therapeutic effect caused by use of the photosensitizer can be simultaneously obtained.

In addition, the photosensitizer-fucoidan conjugate according to the present invention not only may exhibit fluorescence imaging diagnostic efficacy for cancer cells, atherosclerotic plaques, and neovascular endothelial cells, but also may effectively inhibit proliferation of smooth muscle cells which is a major factor that causes vascular restenosis.

Accordingly, the present invention relates to a composition for fluorescence imaging diagnosis or a composition for photodynamic therapy, comprising the photosensitizer-fucoidan conjugate.

In the present invention, the coupling agent refers to a reagent capable of promoting or forming a bond between two or more functional groups which are intramolecularly, intermolecularly, or both present. In the present invention, as the coupling agents, N-(3-dimethylaminopropyl)-N′-ethylcarbodiimide hydrochloride (EDC) and N-hydroxysulfosuccinimide sodium salt (sulfo-NHS) may be preferably used.

In preparing fluorescent dye-fucoidan and photosensitizer-fucoidan conjugates, a coupling agent may be used to convert a carboxyl group of fucoidan to a functional group such as amine, thiol, azide, or alkyne, and then the fucoidan may be used to form a conjugate with a fluorescent dye or a photosensitizer. For example, in a case where an alkyne or azide group is introduced into fucoidan, a fluorescent dye or a photosensitizer may be conjugated thereto through click chemistry which is a reaction between azide and alkyne.

In the present invention, for the photosensitizer used in conjugates for photodynamic diagnosis or therapy, photosensitizers which are applicable by those skilled in the art may use be applied. For example, the photosensitizer may be selected among, but is not limited to, the group consisting of: a porphyrin-based compound selected from the group consisting of porphyrins, chlorins, pheophorbides, bacteriochlorins, porphycenes, and phthalocyanines; and a non-porphyrin-based compound selected from the group consisting of hypericin, rhodamine, rose Bengal, psoralen, phenothiazinium-based dyes, and merocyanine More specifically, the photosensitizers may be used alone or in combination, which are selected from the group consisting of: a porphyrin-based compound selected from the group consisting of hematoporphyrins, porphycenes, pheophorbides, purpurins, chlorins, protoporphyrins, and phthalocyanines, in the form of free bases or metal complexes; and a non-porphyrin-based compound selected from the group consisting of hypericin, rhodamine, ATTO, rose Bengal, psoralen, phenothiazinium-based dyes, and merocyanine.

In the present invention, the linker by which the photosensitizer or the fluorescent dye and the fucoidan are covalently bonded to each other may be a zero-length linker, that is, the liker may contain an amide bond in which an amine group of the fluorescent dye or the photosensitizer is linked to a carboxyl group of the fucoidan, a carbon-carbon bond, a disulfide bond, or a diselenide bond. The linker according to the present invention may have an amine group at both ends. According to the present invention, the amine group of the linker is covalently bonded to the carboxy group of the fucoidan, to form a fucoidan conjugate to which the linker is covalently bonded.

In an embodiment of the present invention, the linker may be selected from the group consisting of a coupling agent such as EDC-NHS, selenocystamine, diselenodipropionic acid, selenocystine, cystine, cystamine, and mixtures thereof.

In an embodiment of the present invention, the photosensitizer may be chlorin e6 which is a chlorin-based photosensitizer as represented below. A carboxyl group of the chlorine e6 is covalently bonded to the amine group of the fucoidan conjugate to which the linker is covalently bonded, to form a photosensitizer-fucoidan conjugate.

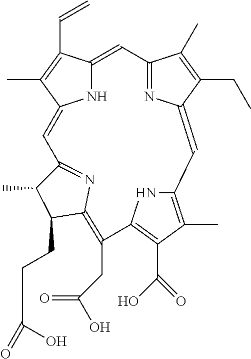 [SMILES: C=Cc1c(C)c2cc3nc(c(CC(=O)O)c4[nH]c(cc5nc(cc1[nH]2)C(C)=C5CC)c(C)c4C(=O)O)[C@@H](CCC(=O)O)[C@@H]3C]

According to the present invention, the fucoidan conjugate to which the linker is covalently bonded is taken up into a target cell, where a disulfide bond or a diselenide bond in the linker is broken by glutathione, a reducing agent that is excessively present inside the cell, so that the photosensitizer or fluorescent dye which has been bound to the fucoidan may be released in the target cell.

The present invention also relates to a composition for photodynamic therapy, comprising the photosensitizer-fucoidan conjugate.

The fluorescent dye- or photosensitizer-fucoidan conjugate of the present invention may specifically target P-selectin overexpressing cells, and thus may exert a therapeutic effect.

Diseases that can be diagnosed/treated with the fluorescent dye- or photosensitizer-fucoidan conjugate according to the present invention include, but is not limited to, tumor diseases such as acral lentiginous melanoma, actinic keratosis, adenocarcinoma, adenoid cystic carcinoma, adenoma, adenosarcoma, adenosquamous carcinoma, astrocytic tumors, glucagonoma, hemangioblastoma, hemangioendothelioma, hemangioma, hepatic adenoma, hepatic adenomatosis, hepatocellular carcinoma, insulinoma, interepithelial neoplasia, interepithelial squamous cell neoplasia, invasive squamous cell carcinoma, large cell carcinoma, leiomyosarcoma, lentigo maligna melanoma, malignant melanoma, malignant mesothelioma, medulloblastoma, and medulloepithelioma, and cancer diseases such as pituitary adenoma, neuroglioma, encephalophyma, nasopharyngeal carcinoma, laryngeal cancer, thymoma, mesothelioma, breast cancer, lung cancer, gastric cancer, esophageal cancer, colorectal cancer, hepatoma, pancreatic cancer, intrapancreatic secreting-tumor, gallbladder cancer, penile cancer, ureteral cancer, renal cell carcinoma, prostate cancer, bladder cancer, non-hodgkin's lymphoma, myelodysplastic syndrome, multiple myeloma, plasma cell neoplasm, leukemia, childhood cancers, skin cancer, ovarian cancer, and cervical cancer. Other diseases that can be treated with the fluorescent dye- or photosensitizer-fucoidan conjugate according to the present invention include sickle cell disease, arterial thrombosis, rheumatoid arthritis, ischemia and reperfusion, arteriosclerosis plaque, vascular restenosis occurring after stenting, ophthalmic diseases such as senile macular degeneration and glaucoma, acne, and dental diseases.

The composition for fluorescence imaging diagnosis or the composition for photodynamic therapy, according to the present invention, may further comprise a pharmaceutically acceptable carrier. The pharmaceutically acceptable carrier is one typically used in formulation, and includes, but is not limited to, lactose, dextrose, sucrose, sorbitol, mannitol, starch, gum acacia, calcium phosphate, alginate, gelatin, calcium silicate, microcrystalline cellulose, polyvinylpyrrolidone, cellulose, water, syrup, methyl cellulose, methyl hydroxybenzoate, propyl hydroxybenzoate, talc, magnesium stearate, mineral oil, and the like. The composition for photodynamic therapy of the present invention may further comprise, in addition to the above ingredients, a lubricant, a humectant, a sweetener, a flavor, an emulsifier, a suspending agent, a preservative, and the like.

The composition for fluorescence imaging diagnosis or the composition for photodynamic therapy, according to the present invention, may be formulated using methods known in the art. The formulation may be in the form of powders, granules, tablets, emulsions, syrups, aerosols, soft or hard gelatin capsules, sterile injectable solutions, or sterile powders.

In addition, the composition for fluorescence imaging diagnosis or the composition for photodynamic therapy, according to the present invention, may be administered orally or parenterally depending on a desired method, and a dose thereof may vary appropriately depending on the patient's body weight, age, sex, health condition, diet, time of administration, mode of administration, excretion rate, severity of disease, and the like.

The fucoidan-fluorescent dye conjugate or the fucoidan-photosensitizer conjugate, according to the present invention, allows the photosensitizer or fluorescent dye to be dissolved well in water, and allows fluorescence imaging and photodynamic therapy to be effectively achieved. In addition, such a conjugate may exhibit a therapeutic effect due to the fucoidan polymer itself or may exhibit a target specificity effect on P-selectin overexpressing cells. According to the present invention, the fucoidan polymer itself exhibits direct cytotoxicity against cancer cells or smooth muscle cells, or an effect of inhibiting cancer growth or inhibiting neovascularization in ophthalmic diseases is further obtained due to binding of the fucoidan with vascular endothelial growth factor (VEGF), so that an improved therapeutic effect, which could not be expected from the existing photosensitizers or fluorescent dyes and hydrophilic polymers, can be obtained.

The photosensitizer-fucoidan conjugate according to the present invention not only may exhibit fluorescence imaging diagnostic efficacy for cancer cells, atherosclerotic plaques, and neovascular endothelial cells, but also may exhibit, in a simultaneous manner, a therapeutic effect caused by the fucoidan itself and a photodynamic therapeutic effect caused by use of the photosensitizer. At the site where a stent is mounted for vasodilation, vascular restenosis occurs over time due to proliferation of smooth muscle cells. However, in a case where the photosensitizer-fucoidan conjugate according to the present invention is used, it is possible to effectively inhibit proliferation of smooth muscle cells which is a major factor that causes vascular stenosis.

Since the fluorescent dye-fucoidan conjugate according to the present invention can retain high binding specificity for P-selectin and vascular endothelial growth factor even after formation of the conjugate, such a conjugate has an advantage that it not only allows for fluorescence imaging diagnosis of neovascularization sites in cancer cells, atherosclerotic plaques, and ophthalmic diseases, but also may exhibit a therapeutic effect due to the fucoidan.

The fluorescent dye-fucoidan conjugate and the photosensitizer-fucoidan conjugate, according to the present invention, are not only useful for fluorescence imaging diagnosis of tumor tissues and ophthalmic vascular diseases, but also may exhibit a therapeutic effect on cancer cells and coronary artery smooth muscle cells, and a neovascularization inhibitory effect in ophthalmic diseases. In addition, in a case where photodynamic therapy is further implemented on the photosensitizer-fucoidan conjugate, cancer may be effectively treated with low adverse effects.

### BRIEF DESCRIPTION OF THE DRAWINGS

The patent or application file contains at least one drawing executed in color. Copies of this patent or patent application publication with color drawings will be provided by the Office upon request and payment of the necessary fee.

FIG. 1 illustrates a schematic diagram of a fucoidan-based fluorescent dye or photosensitizer conjugate. The photosensitizer or fluorescent dye is covalently bonded to fucoidan via a linker, and the linker may contain an amide bond, a carbon-carbon bond, a disulfide bond, or a diselenide bond.

FIG. 2 illustrates a schematic diagram for synthesis of a conjugate of a fluorescent dye having an amine group with fucoidan. The fluorescent dye-fucoidan conjugate was prepared using EDS and NHS which are coupling agents.

FIG. 3A and FIG. 3B illustrate data obtained by identifying optical properties of the prepared Flamma774-Fucoidan conjugate, through UV-Vis absorbance (A) and fluorescence spectrum (B).

FIG. 4A and FIG. 4B illustrate data obtained by identifying, through FT-IR analysis, fucoidan (FIG. 4A) and the prepared Flamma774-Fucoidan conjugate (FIG. 4B).

FIG. 5A and FIG. 5B illustrate data obtained by identifying, through ¹H-NMR analysis, fucoidan (FIG. 5A) and the prepared Flamma774-Fucoidan conjugate (FIG. 5B).

FIG. 6A and FIG. 6B illustrate data obtained by identifying optical properties of the prepared ATTO655-Fucoidan conjugate, through UV-Vis absorbance (FIG. 6A) and fluorescence spectrum (FIG. 6B).

FIG. 7 illustrates a schematic diagram for a method of synthesizing a ZW800-Fucoidan conjugate by causing ZW800 dye, a near-infrared fluorescent dye, to be bound to fucoidan.

FIG. 8A illustrates data obtained by identifying, through UV-Vis absorbance and fluorescence spectrum, optical properties of the ZW800-Fucoidan conjugate in a case where reaction is allowed to proceed at 1:2 depending on a ratio.

FIG. 8B illustrates data obtained by identifying, through UV-Vis absorbance and fluorescence spectrum, optical properties of the ZW800-Fucoidan conjugate in a case where reaction is allowed to proceed at 1:4 depending on a ratio.

FIG. 9A, FIG. 9B, and FIG. 9C illustrate data obtained by identifying, through surface plasmon resonance (SPR) analysis, binding affinity between the prepared ZW800-Fucoidan conjugates and vascular epithelial growth factor (VEGF165) ligand, and binding affinity between fucoidan itself and vascular epithelial growth factor (VEGF165) ligand.

FIG. 10A, FIG. 10B, and FIG. 10C illustrate data obtained by identifying, through surface plasmon resonance (SPR) analysis, binding affinity between the prepared ZW800-Fucoidan conjugates and P-selectin, and binding affinity between fucoidan itself and P-selectin.

FIG. 11A and FIG. 11B illustrate data obtained by identifying optical properties of the prepared FSD750-Fucoidan conjugate, through UV-Vis absorbance (FIG. 11A) and fluorescence spectrum (FIG. 11B).

FIG. 12 illustrates a schematic diagram of synthesis, showing that chlorin e6 (Ce6), a photosensitizer, is bound to fucoidan via a linker containing a disulfide bond (—SS—), to form a Ce6-Fucoidan conjugate, and the resultant is subjected to self-assembly so that nanometer-sized nanoparticles for photodynamic diagnosis and therapy are obtained.

FIG. 13A illustrates a graph, showing size distribution (hydrodynamic size) in aqueous solution of the prepared Ce6-Fucoidan (fucoidan molecular weight of 18 kDa) conjugate.

FIG. 13B illustrates UV-Vis absorbance spectra of the prepared Ce6-Fucoidan (18 kDa) conjugate depending on solvents.

FIG. 13C illustrates fluorescence spectra of the prepared Ce6-Fucoidan (18 kDa) conjugate depending on solvents.

FIG. 14A illustrates a graph, showing size distribution (hydrodynamic size) in aqueous solution of the prepared Ce6-Fucoidan (fucoidan molecular weight of 100 kDa) conjugate.

FIG. 14B illustrates a photograph obtained by analyzing morphology of the prepared Ce6-Fucoidan (100 kDa) conjugate with a transmission electron microscope.

FIG. 15A illustrates UV-Vis absorbance spectra of the prepared Ce6-Fucoidan (100 kDa) conjugate depending on solvents.

FIG. 15B illustrates fluorescence spectra of the prepared Ce6-Fucoidan (100 kDa) conjugate depending on solvents.

FIG. 15C illustrates fluorescence spectra obtained by subjecting the prepared Ce6-Fucoidan (100 kDa) to treatment with glutathione (GSH) at concentrations of 0 μM, 5 μM, and 5 mM, respectively.

FIG. 15D illustrates data obtained by subjecting the prepared Ce6-Fucoidan (100 kDa) to treatment with glutathione (GSH) at concentrations of 0 μM, 5 μM, and 5 mM, respectively, for 4 hours, and measuring production of singlet oxygen under irradiation with light of 670 nm. As single oxygen is generated, fluorescence of singlet-oxygen-detecting reagent (SOSG) increases.

FIG. 16A and FIG. 16B illustrate results of ¹H-NMR analysis for a free photosensitizer (free Ce6) and the prepared Ce6-Fucoidan conjugate.

FIG. 17 illustrates confocal fluorescence micrographs obtained after subjecting cancer cells to treatment with Ce6-Fucoidan, a photosensitizer-fucoidan conjugate, and a free photosensitizer (free Ce6) at the same concentration. It was identified that the Ce6-Fucoidan conjugate can be taken up much better into cancer cells.

FIG. 18A illustrates results obtained by subjecting cancer cells to treatment with the Ce6-Fucoidan conjugate and a free photosensitizer at various concentrations, and analyzing cell viability. It can be seen that a therapeutic effect can be obtained by the conjugate itself without light irradiation.

FIG. 18B illustrates results obtained by subjecting cancer cells to treatment with the Ce6-Fucoidan conjugate and the free photosensitizer, and then analyzing cell viability when photodynamic therapy is performed using a 670 nm laser. Light irradiation made it possible to obtain a very improved cancer therapeutic effect.

FIG. 19A illustrates near-infrared fluorescence imaging results obtained 5 minutes and 24 hours after intravenous injection of the Ce6-Fucoidan conjugate. It can be seen that in a case where the Ce6-Fucoidan conjugate is administered, location of a cancer tissue can be diagnosed from the fluorescence image.

FIG. 19B illustrates results obtained by quantitative analysis of tumor-to-background signal ratio values.

FIG. 19C illustrates results obtained by collecting tumors and major organs 24 hours after administration of the Ce6-Fucoidan conjugate and taking fluorescence images thereof. It can be seen that the Ce6-Fucoidan conjugate is accumulated in tumor tissues.

FIG. 19D illustrates results obtained by excising tumors to prepare frozen sections 24 hours after administration of the Ce6-Fucoidan conjugate and taking fluorescence images thereof with a confocal microscope. It can be seen that the photosensitizer is present at a higher concentration in the tumors having received the Ce6-Fucoidan conjugate.

FIG. 20A illustrates results, showing an antitumor effect caused by use of the Ce6-Fucoidan conjugate, a photosensitizer-fucoidan conjugate. It can be seen that intravenous administration of the Ce6-Fucoidan conjugate can achieve a significant anticancer effect even in a case where light irradiation is not used; and it can be seen that a very high cancer therapeutic effect can be obtained in a case where tumors are irradiated with light.

FIG. 20B illustrates results obtained by excising tumor tissues to prepare sections 24 hours after combined treatment of the Ce6-Fucoidan conjugate and photodynamic therapy, identifying vascular distribution in the tumor tissues with CD31 staining, and identifying a cell apoptosis effect with TUNEL staining.

FIG. 21A illustrates results obtained by extracting major organs from mice of each experimental group on Day 10 and identifying toxicity through H&E staining.

FIG. 21B illustrates results obtained by measuring changes in body weight of mice for each experimental group.

FIG. 22A illustrates results obtained by subjecting coronary smooth muscle cells to treatment with the Ce6-Fucoidan conjugate and a free photosensitizer at various concentrations, and analyzing cell viability.

FIG. 22B illustrates results obtained by subjecting coronary smooth muscle cells to treatment with the Ce6-Fucoidan conjugate and a free photosensitizer at various concentrations, implementing photodynamic therapy using a 670 nm laser, and then analyzing cell viability.

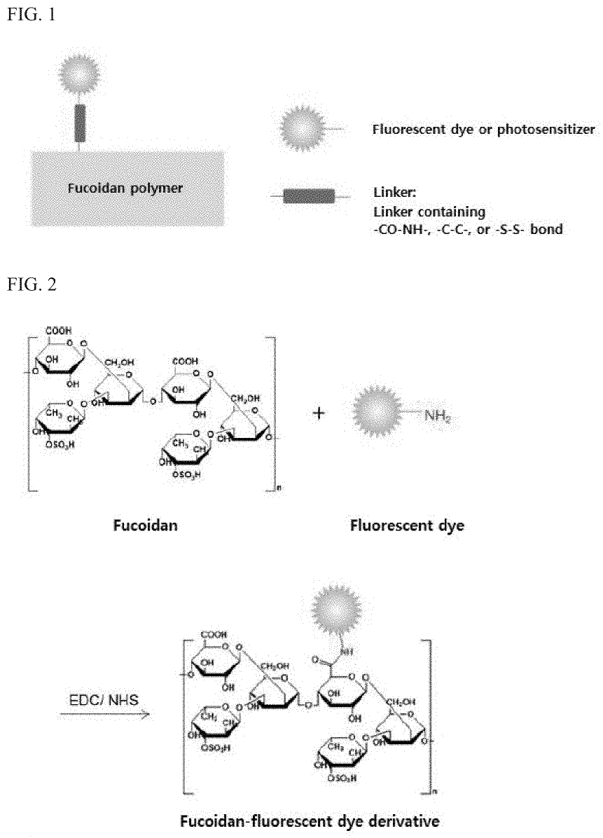

### DESCRIPTION OF THE PREFERRED EMBODIMENTS

Hereinafter, in order to describe the present invention in more detail, preferred embodiments of the present invention will be described in more detail with reference to the accompanying drawings. However, the present invention is not limited to the embodiments described herein and may be embodied in other forms. Unless defined otherwise, all technical and scientific terms used herein have the same meaning as commonly understood by those skilled in the art to which the present invention belongs. In general, the nomenclature used herein is well known and commonly used in the art.

### EXAMPLE 1

### Preparation of Flamma774-Fucoidan Conjugate

An amine group of Flamma774, a near-infrared fluorescent dye from BioActs, and a carboxy group of fucoidan were bound to each other using a coupling agent, to synthesize a covalent conjugate. Flamma774-amine is a fluorescent substance having a molar mass of 971.15 g/mol, a maximum excitation wavelength of 774 nm, and a maximum emission wavelength of 806 nm. A near-infrared fluorescent dye conjugate may be used for bioimaging in drug delivery, tumor research, and the like due to its high permeability to biological tissues. Fucoidan was a product of Sigma Aldrich with a molecular weight of 18,000 Da, extracted from Fucus vesiculosus.

Various coupling agents may be used to bind the fluorescent dye to the fucoidan. Here, the following process was used. N-(3-dimethylaminopropyl)-N′-ethylcarbodiimide hydrochloride (EDC) and N-hydroxysulfosuccinimide sodium salt (sulfo-NHS) were used to activate the fucoidan, thereby obtaining a fucoidan-NHS ester combination; and Flamma774-amine was allowed to bind thereto. To describe such a process in more detail, 10 mg of fucoidan was dissolved in 0.1 M 2-(N-morpholino) ethanesulfonic acid (MES) buffer, and 19.7 mg of EDC and 2.17 mg of sulfo-NHS were added thereto. Stirring was performed for about 30 minutes. Then, separation was performed using a PD-10 column, 1.02 mg of Flamma774-amine fluorescence was added, and stirring was performed for one day. Then, the resultant was subjected to dialysis against distilled water for one day so that unreacted reactants and by-products were removed, and freeze-dried to give powders so that a fluorescent dye-fucoidan conjugate in which Flamma774 is covalently bonded to fucoidan was obtained.

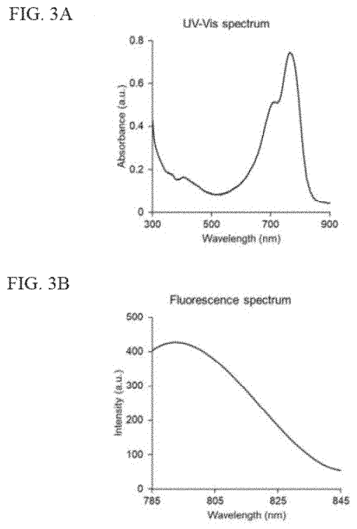

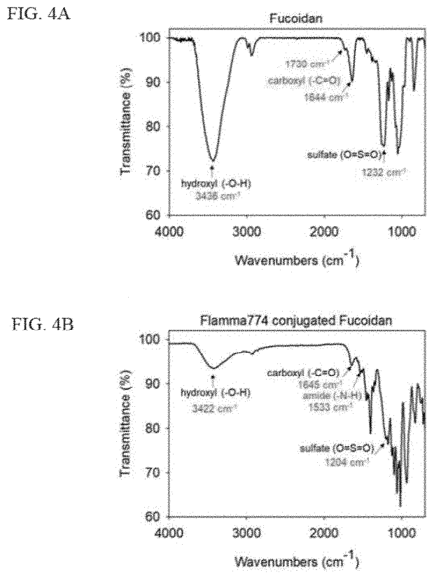

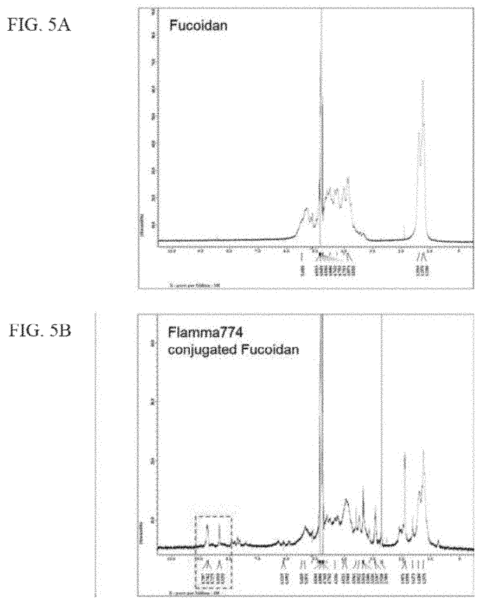

It was identified through optical measurement analysis of FIG. 3A and FIG. 3B that about 3.8 Flamma774 molecules are bound per molecule of fucoidan.

### EXAMPLE 2

### Preparation of ATT0655-Fucoidan Conjugate

An amine group of ATTO655, a fluorescent dye, and a carboxy group of fucoidan were bound to each other using a coupling agent, to form a covalent conjugate. ATTO655-amine is a fluorescent substance having a molar mass of 798 g/mol, a maximum excitation wavelength of 663 nm, and a maximum emission wavelength of 680 nm. Fucoidan was a product of Sigma Aldrich with a molecular weight of 18,000 Da, extracted from Fucus vesiculosus.

The following process was used. N-(3-dimethylaminopropyl)-N′-ethylcarbodiimide hydrochloride (EDC) and N-hydroxysulfosuccinimide sodium salt (sulfo-NHS) were used to activate the fucoidan, thereby obtaining a fucoidan-NHS ester combination; and ATTO655-amine was allowed to bind thereto. To describe such a process in more detail, 10 mg of fucoidan was dissolved in 0.1 M 2-(N-morpholino) ethanesulfonic acid (MES) buffer, and 19.7 mg of EDC and 2.17 mg of sulfo-NHS were added thereto. Stirring was performed for about 30 minutes. Then, separation was performed using a PD-10 column, 0.399 mg of ATTO655-amine fluorescence was added, and stirring was performed for one day. Then, the resultant was subjected to dialysis against distilled water for one day, and freeze-dried to give powders so that a fluorescent dye-fucoidan conjugate in which ATTO655-amine is covalently bonded to fucoidan was obtained.

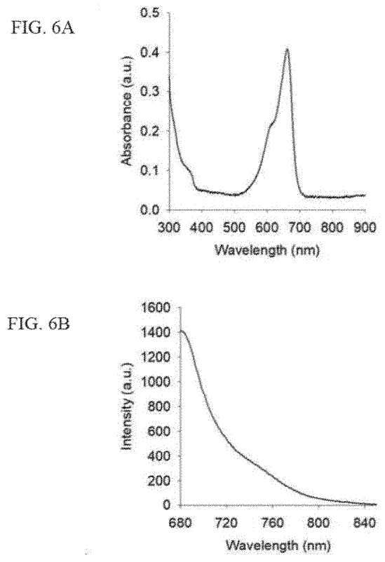

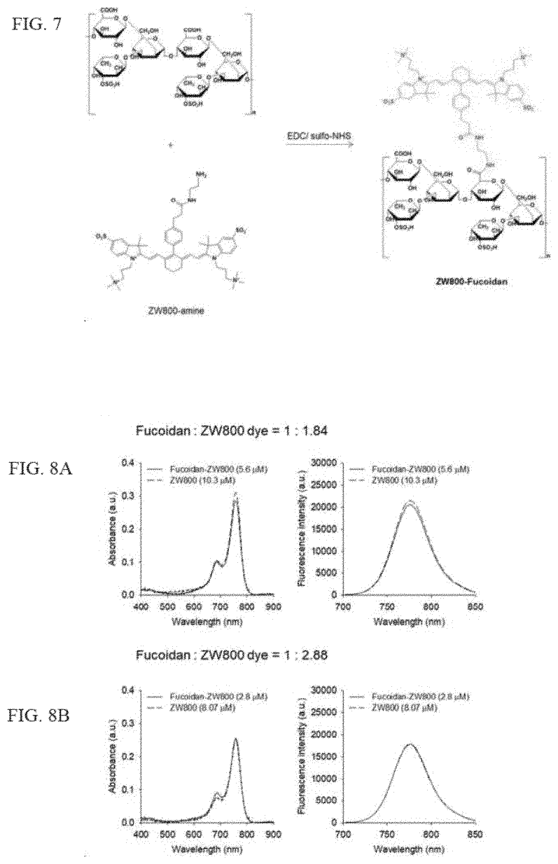

It was identified through optical measurement analysis of FIG. 6A and FIG. 6B that about 1.3 ATTO dye molecules are bound per molecule of fucoidan.

### EXAMPLE 3

### Preparation of ZW800-Fucoidan Conjugate

An amine group of ZW800, a near-infrared fluorescent dye developed by a research team led by Professor Hak Soo CHOI at Harvard Medical School, and a carboxy group of fucoidan were bound to each other using a coupling agent, to form a covalent conjugate. ZW800-amine is a near-infrared fluorescent substance having a molar mass of 887 g/mol, a maximum excitation wavelength of 753 nm, and a maximum emission wavelength of 772 nm. Fucoidan was a product of Sigma Aldrich with a molecular weight of 18,000 Da, extracted from Fucus vesiculosus.

The following process was used. N-(3-dimethylaminopropyl)-N′-ethylcarbodiimide hydrochloride (EDC) and N-hydroxysulfosuccinimide sodium salt (sulfo-NHS) were used to activate the fucoidan, thereby obtaining a fucoidan-NHS ester combination; and ZW800-amine was allowed to bind thereto. The fucoidan and the ZW800 fluorescent dye were reacted at reaction ratios of 1:2 and 1:4, respectively. To describe such a process in more detail, 20 mg of fucoidan was dissolved in 0.1 M 2-(N-morpholino) ethanesulfonic acid (MES) buffer, and 38.34 mg of EDC and 4.34 mg of sulfo-NHS were added thereto. Stirring was performed for about 30 minutes. Then, separation was performed using a PD-10 column; 1.97 mg of ZW800-amine fluorescence was added in a case where the fucoidan and the ZW800 fluorescent dye are reacted at a reaction ratio of 1:2, and 3.94 mg of ZW800-amine fluorescence was added in a case where the fucoidan and the ZW800 fluorescent dye are reacted at a reaction ratio of 1:4; and stirring was performed for one day. Then, the resultant was subjected to dialysis against distilled water for one day and freeze-dried to give powders so that a fluorescent dye- fucoidan conjugate in which ZW800 is covalently bonded to fucoidan was obtained.

It was identified through optical measurement analysis of FIG. 8A and FIG. 8B that in a case where the fucoidan and the ZW800 fluorescent dye are reacted at a reaction ratio of 1:2, about 1.84 ZW800 fluorescent dye molecules are bound per molecule of fucoidan. It was identified that in a case where the fucoidan and the ZW800 fluorescent dye are reacted at a reaction ratio of 1:4, about 2.88 ZW800 fluorescent dye molecules are bound per molecule of fucoidan.

### EXAMPLE 4

### Binding Affinity of ZW800-Fucoidan Conjugate and VEGF165 Ligand

Binding affinity between the synthesized ZW800-Fucoidan conjugate and human VEGF165 ligand was analyzed using surface plasmon resonance (SPR). SPR sensor technique uses a phenomenon, in which a signal change is caused in a case where a biological material such as a protein is bound onto the sensor surface, and is an analytical method in which the SPR optical principle is used to measure correlation (kinetics affinity, Ka, Kd, KD) between biological molecules in real time without specific labels (fluorescence, radioactivity, and the like). Analysis was performed using, as SPR analysis equipment, Biacore T200 equipment and CMS chip, and then data was processed by Biaevaluation software.

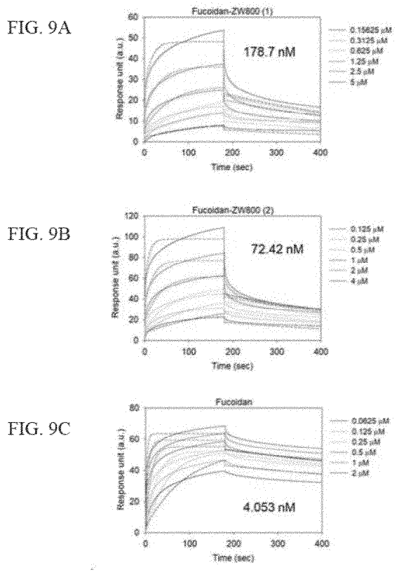

FIGS. 9A, 9B, and 9C illustrate results obtained by analyzing, through equilibrium dissociation rate constant (KD) values of SPR assay, binding affinity between ZW800-Fucoidan conjugates (1) and (2), where the fucoidan and the ZW800 fluorescent dye were reacted at reaction ratios of 1:2 and 1:4, respectively, and human VEGF165 ligand, and binding affinity between fucoidan having no label and human VEGF165 ligand. The binding affinity was measured as 178.7 nM for the ZW800-Fucoidan conjugate with a reaction ratio of 1:2, as 72.42 nM for the ZW800-Fucoidan conjugate with a reaction ratio of 1:4, and as 4.053 nM for the fucoidan. It was found that these results show strong binding, in unit of 10⁻⁹ M, to VEGF even after formation of the fucoidan-fluorescent conjugate. Therefore, the fluorescent dye-fucoidan conjugate not only allows for fluorescence imaging diagnosis of vascular diseases, but also can exhibit a neovascularization inhibitory effect in ophthalmic diseases and the like through binding of the fluorescent dye-fucoidan conjugate to VEGF.

### EXAMPLE 5

### Binding Affinity between ZW800-Fucoidan Conjugate and P-selectin

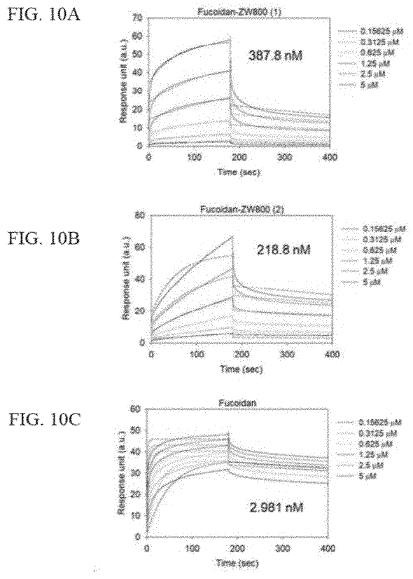

FIGS. 10A, 10B, and 10C illustrate results obtained by analyzing, through equilibrium dissociation rate constant (KD) values of SPR assay, binding affinity between ZW800-Fucoidan conjugates (1) and (2), where fucoidan and ZW800 fluorescent dye were reacted at reaction ratios of 1:2 and 1:4, respectively, and P-selectin, and binding affinity between fucoidan having no label and P-selectin. The binding affinity was measured as 387.8 nM for the ZW800-Fucoidan conjugate with a reaction ratio of 1:2, as 218.8 nM for the ZW800-Fucoidan conjugate with a reaction ratio of 1:4, and as 2.981 nM for the fucoidan. It was found that these results show strong binding, in unit of 10⁻⁹ M, to P-selectin even after formation of the fucoidan-fluorescent conjugate. These results indicate that the fluorescent dye-fucoidan conjugate can be used to diagnose, with images, lesions for vascular diseases such as metastatic cancer cells or atherosclerotic plaques which overexpress P-selectin on the cell surface.

### EXAMPLE 6

### Preparation of FSD750-Fucoidan Conjugate

An amine group of FSD750, a near-infrared fluorescent substance from BioActs, and a carboxy group of fucoidan were covalently bonded to each other so that a covalent conjugate can be formed. FSD750-amine is a fluorescent substance having a molar mass of 1252.42 g/mol, a maximum excitation wavelength of 749 nm, and a maximum emission wavelength of 774 nm. Fucoidan was a product of Sigma Aldrich with a molecular weight of 18,000 Da, extracted from Fucus vesiculosus.

The following process was used. N-(3-dimethylaminopropyl)-N′-ethylcarbodiimide hydrochloride (EDC) and N-hydroxysulfosuccinimide sodium salt (sulfo-NHS) were used to activate the fucoidan, thereby obtaining a fucoidan-NHS ester combination; and FSD750-amine was allowed to bind thereto. To describe such a process in more detail, 5 mg of fucoidan was dissolved in 0.1 M 2-(N-morpholino) ethanesulfonic acid (MES) buffer, and 9.585 mg of EDC and 1.085 mg of sulfo-NHS were added thereto. Stirring was performed for about 30 minutes. Then, separation was performed using a PD-10 column, 0.695 mg of FSD750-amine fluorescence was added, and stirring was performed for one day. Then, the resultant was subjected to dialysis against distilled water for one day and freeze-dried to give powders so that a fluorescent dye-fucoidan conjugate in which FSD750 is covalently bonded to fucoidan was obtained.

It was identified through optical measurement analysis of FIGS. 11A and 11B that about 1.2 FSD750 fluorescent dye molecules are bound per molecule of fucoidan.

### EXAMPLE 7

### Preparation of Photosensitizer-Fucoidan (18 kDa) Conjugate

Chlorin e6 (Ce6), a photosensitizer, and a carboxyl group of fucoidan were covalently bonded to each other via a linker, to synthesize a photosensitizer-fucoidan conjugate. Fucoidan was a product of Sigma Aldrich with a molecular weight of 18,000 Da, extracted from Fucus vesiculosus.

First, in order to synthesize fucoidan into which an amine group is introduced, cystamine dihydrochloride, a linker containing a disulfide bond, was covalently bonded to a carboxyl group of fucoidan using EDC and sulfo-NHS. To describe such a process in more detail, 54.36 mg of fucoidan was dissolved in 18 mL of 10 mM PBS buffer, and 23.0 mg (0.5 mL) of EDC and 27.1 mg (0.5 mL) of sulfo-NHS were added thereto. Stirring was performed for about 30 minutes. Then, 27 mg (1 mL) of cystamine dihydrochloride, a linker containing a disulfide bond, was added thereto, and stirring was performed for one day. Then, the resultant was subjected to dialysis against distilled water for one day and freeze-dried to give powders so that a fucoidan derivative having an amine group was obtained.

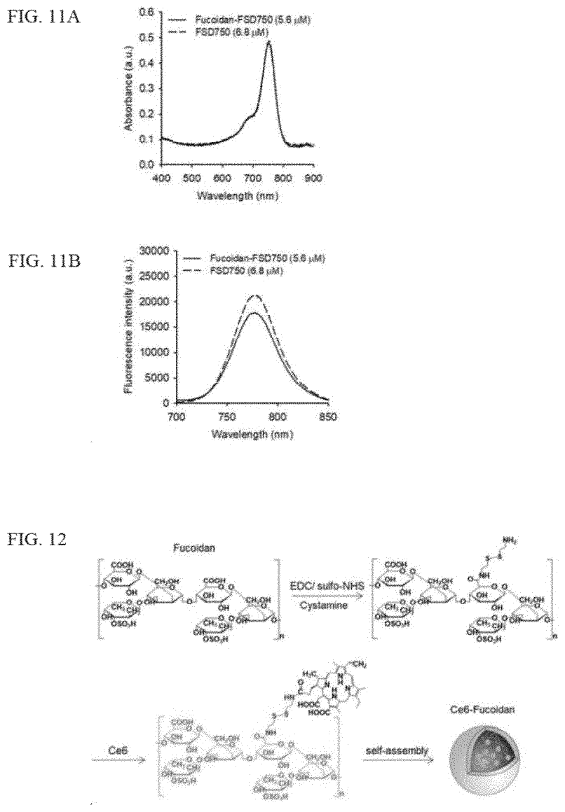

The following process was used. EDC and sulfo-NHS were used to activate a carboxy group of Ce6, and then the fucoidan into which an amine group is introduced was allowed to bind thereto. 5 mg of Ce6 was dissolved in 2.5 mL of dimethyl sulfoxide (DMSO), and 16.3 mg of EDC and 19 mg of sulfo-NHS were added thereto. Stirring was performed for one hour. Then, 30.15 mg of fucoidan into which an amine group is introduced was dissolved in 2.5 mL of DMF: H2O co-solvent (1:1 v/v) and mixed with the Ce6 reaction solution. Then, stirring was performed for one day. Then, the resultant was subjected to dialysis for one day using phosphate buffer (pH 7.4) and distilled water, and freeze-dried to give powders so that a photosensitizer-fucoidan conjugate was obtained. Referring to FIG. 13A, it can be seen that the prepared photosensitizer-fucoidan conjugate forms nanoparticles in an aqueous solution. Referring to FIGS. 13B and 13C, it can be seen that fluorescence properties of the prepared conjugate are inhibited.

### EXAMPLE 8

### Preparation of Fucoidan (100 kDa)-Photosensitizer Conjugate

Chlorin e6 (Ce6), a photosensitizer, and a carboxyl group of fucoidan were covalently bonded to each other via a linker, to synthesize a photosensitizer-fucoidan conjugate. The fucoidan used in the preparation of the conjugate was a product of Haerimfucoidan Co., Ltd., which is fucoidan with a molecular weight of 100,000 Da, extracted from Undaria pinnatifida.

First, in order to synthesize fucoidan into which an amine group is introduced, cystamine dihydrochloride, a linker containing a disulfide bond, was covalently bonded to a carboxyl group of fucoidan using EDC and sulfo-NHS. To describe such a process in more detail, 405 mg of fucoidan was dissolved in 18 mL of 10 mM PBS buffer, and 23.0 mg (0.5 mL) of EDC and 27.1 mg (0.5 mL) of sulfo-NHS were added thereto. Stirring was performed for about 30 minutes. Then, 27 mg (1 mL) of cystamine dihydrochloride was added thereto and stirring was performed for one day. Then, the resultant was subjected to dialysis against distilled water for one day and freeze-dried to give powders so that a fucoidan derivative having an amine group was obtained.

The following process was used. EDC and sulfo-NHS were used to activate a carboxy group of Ce6, and then the fucoidan into which an amine group is introduced was allowed to bind thereto. 20 mg of Ce6 was dissolved in 10 mL of DMSO, and 65.2 mg of EDC and 76 mg of sulfo-NHS were added thereto. Stirring was performed for one hour. Then, 101 mg of fucoidan into which an amine group is introduced was dissolved in 5 mL of DMF: H2O co-solvent (1:1 v/v), and mixed with the Ce6 reaction solution. Then, stirring was performed for one day. Then, the resultant was subjected to dialysis for one day using phosphate buffer (pH 7.4) and distilled water, and freeze-dried to give powders so that a photosensitizer-fucoidan conjugate was obtained.

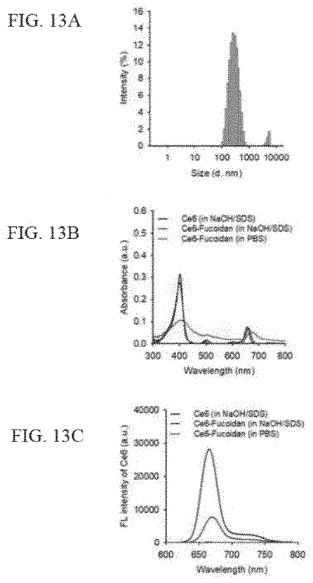

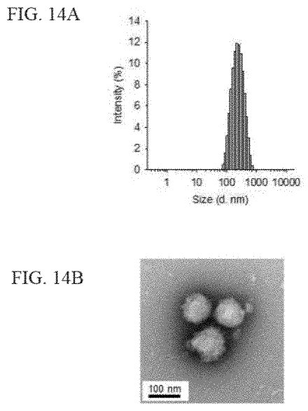

FIG. 14A illustrates a result obtained by measuring, with a particle size analyzer, an average particle size of the photosensitizer-fucoidan conjugate as prepared above, and the average size was determined to be 259 nm. FIG. 14B illustrates a result obtained by analyzing the photosensitizer-fucoidan conjugate with a transmission electron microscope. As illustrated, it can be seen that nanoparticles with a size of about 85 nm were obtained.

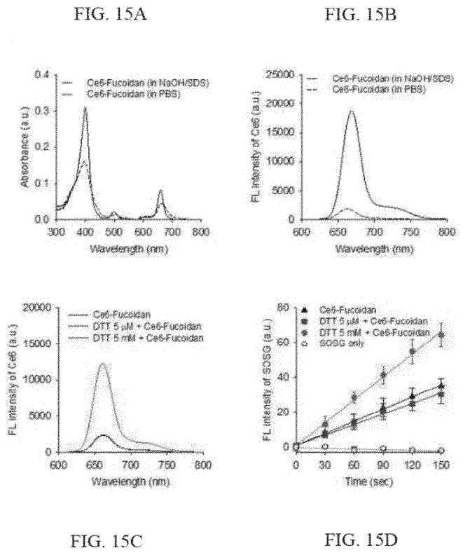

The prepared photosensitizer-fucoidan conjugate was dissolved in NaOH/SDS mixed solution, which serves as surfactant, and phosphate buffered saline (PBS) solution (0.1 M, pH 7.4), and then the amount of Ce6 bound was analyzed through absorbance. FIGS. 15A and 15B illustrate UV-vis absorbance and fluorescence spectra.

In addition, in order to check whether, as the prepared Ce6-Fucoidan (100 kDa) is subjected to treatment with glutathione (GSH) at concentrations of 0 μM, 5 μM, and 5 mM, respectively, a disulfide bond is broken and thus the quenched derivative exhibits changed Ce6 fluorescence intensity, changes in fluorescence intensity depending on treatment concentrations of glutathione were measured. FIG. 15C illustrates a fluorescence spectrum observed therefor. It was identified that fluorescence intensity does not change in a case where Ce6-Fucoidan (100 kDa) is subjected to treatment with glutathione at a concentration of 5 μM at which glutathione is present in cells other than cancer cells or in the blood, whereas about 5-fold increase in fluorescence intensity is observed in a case of being subjected to treatment with glutathione at a concentration of 5 mM at which glutathione is present in cancer cells.

FIG. 15D illustrates results obtained by subjecting the prepared Ce6-Fucoidan (100 kDa) to treatment with glutathione (GSH) at concentrations of 0 μM, 5 μM, and 5 mM, respectively, for 4 hours, and analyzing production of singlet oxygen at 30-second intervals under light irradiation with a 670 nm laser. It was identified that in a case of being subjected to treatment with glutathione at a concentration of 5 mM at which glutathione is present in cancer cells, about 2-fold increase in production of singlet oxygen is observed. These results indicate that in a case where a linker containing a disulfide bond is used, the conjugate is taken up into target cancer cells, and that in a case where the disulfide bond is decomposed by glutathione, a reducing agent, present at a high concentration in cancer cells, fluorescence generation and photodynamic therapeutic effects can be restored again in a cancer cell-specific manner.

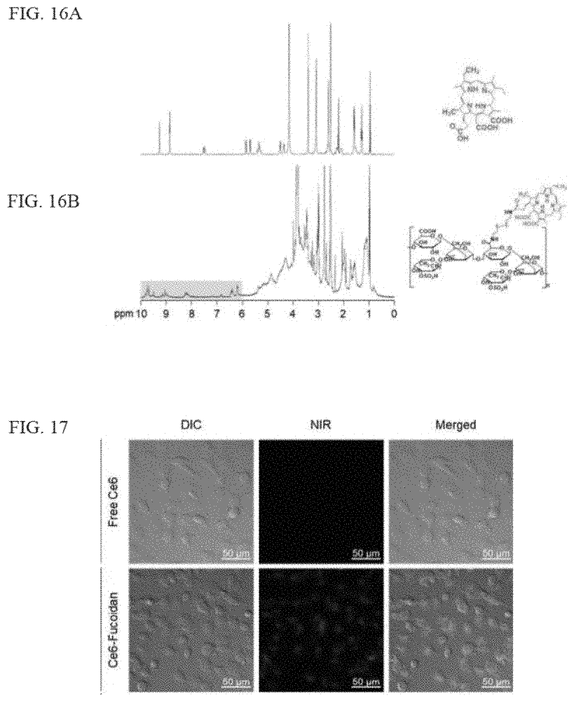

FIG. 16A illustrates a ¹H-NMR analysis result for Ce6, a photosensitizer, and FIG. 16B illustrates a ¹H-NMR analysis result for the photosensitizer-fucoidan conjugate. Through the ¹H-NMR analyses, it was calculated that about 20 Ce6 molecules are bound per molecule of fucoidan.

### EXAMPLE 9

### Identification of Uptake of Photosensitizer-Fucoidan Conjugate by Cancer Cells

Degree of uptake of a photosensitizer-fucoidan conjugate and a free photosensitizer (free Ce6) by HT1080 cells, which are cancer cells, was compared by a confocal fluorescence microscope.

1) Cell Culture

HT1080, a human fibrosarcoma cell line, was obtained from the American Type Culture Collection (ATCC, USA). The HT1080 cells were cultured, under conditions of 37° C., 5% carbon dioxide, and standard humidity, in Eagle's Minimum Essential Media (MEM) medium supplemented with 10% fetal bovine serum (FBS) and 1% penicillin/streptomycin.

2) Experiment for Identifying Cellular Uptake

The HT1080 cells were placed at 5×10⁴ in each well of LabTek II Chambered Coverglass and incubated for 24 hours so that the cells adhere well thereto. The cancer cells were subjected for 6 hours to treatment with Ce6-Fucoidan, a photosensitizer-fucoidan conjugate, and a free photosensitizer (free Ce6) at a concentration of 2 μM on a Ce6 basis. Then, the drug which was not taken up into the cells was removed by washing, and a fresh cell culture medium was added thereto. Subsequently, the amount taken up into the cells was compared by a confocal fluorescence microscope (observation condition=excitation: 633 nm, emission: 650 nm long-pass filter). Referring to FIG. 17, it was analyzed that about 18-fold higher fluorescence intensity is observed in the cancer cells treated with the Ce6-Fucoidan, as compared with the cancer cells treated with Ce6. From this, it can be seen that a photosensitizer-fucoidan conjugate can be very effectively taken up into cancer cells as compared with a photosensitizer; and it can be also seen that optical properties which have been quenched are restored in cancer cells.

### EXAMPLE 10

### Analysis of Therapeutic Performance of Photosensitizer-Fucoidan Conjugate Against Cancer Cells

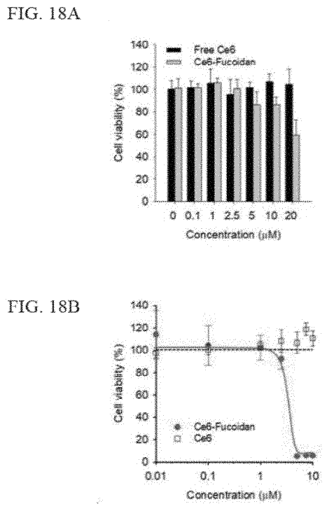

FIG. 18A illustrates results obtained by measuring cell viability depending on concentrations of a photosensitizer-fucoidan conjugate and a free photosensitizer used to treat cancer cells. It can be seen that unlike other biocompatible polymers, fucoidan itself has an anticancer effect.

FIG. 18B illustrates a photodynamic therapeutic effect of Ce6-Fucoidan, a photosensitizer-fucoidan conjugate, on HT1080 cancer cells. A wavelength used for photodynamic therapy was 670 nm, and light intensity was 10 J/cm². The HT1080 cancer cells were subjected for 6 hours to treatment with Ce6, a control, and the Ce6-Fucoidan conjugate according to the present invention at various concentrations. Then, the medium was replaced with fresh MEM medium, and a 670 nm laser was used to perform light irradiation at 10 J/cm². After incubation for additional 24 hours, cell viability was analyzed using a CCK-8 assay kit. As illustrated, it can be seen that the Ce6-Fucoidan conjugate exhibits much better phototoxicity against the HT1080 cancer cells than the conventional Ce6, and it was analyzed that the concentration (IC₅₀) value of substance required to kill about half of the cells is 2.73 μM.

### EXAMPLE 11

### Tumor-Targeting Effect of Photosensitizer-Fucoidan Conjugate

In Example 11, in order to analyze a tumor-targeting effect of a photosensitizer-fucoidan conjugate, an experiment was performed, in which the conjugate is intravenously administered to an experimental animal and a fluorescence image is taken.

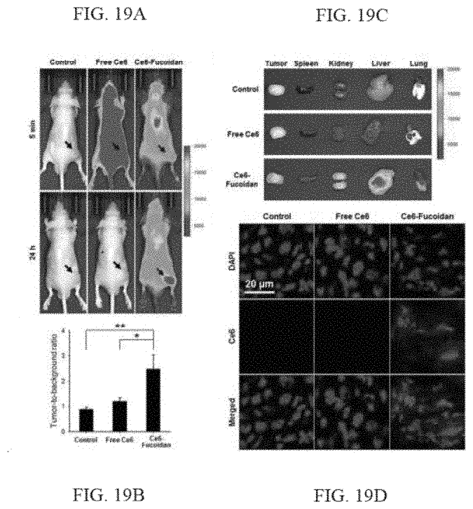

Phosphate buffer (negative control), a free photosensitizer (free Ce6), or a photosensitizer-fucoidan conjugate (Ce6-Fucoidan) was injected intravenously, respectively, into an HT1080 human fibrosarcoma-transplanted animal model, and fluorescence images were taken with an IVIS imaging machine at 5 minutes and 24 hours after the injection (λₑₓ 660/20 nm, λₑₘ 710/40 nm). Referring to the results of FIG. 19A, it was identified that the free photosensitizer (free Ce6) stays short in the body, is excreted out of the body within a short period of time, and hardly remains in the body at 24 hours, so that there is no fluorescence image signal even in tumor tissues. On the other hand, it was identified that the Ce6-Fucoidan conjugate stays long in the body through the blood stream and is continuously accumulated in tumor tissues, so that a clear fluorescence image signal appears in tumor tissues at 24 hours. In FIG. 19B, fluorescence image signal values of the tumor tissues are quantified to compare the amount of photosensitizer accumulated in the tumor tissues. As a result, it was suggested that the photosensitizer-fucoidan conjugate is capable of tumor-targeting, is delivered in a tumor-selective manner, and optical properties thereof are restored again in tumors, thereby inducing an improved therapeutic effect. FIG. 19C illustrates results obtained by allowing mice to be euthanized, obtaining spleen, kidney, liver, and lung tissues, and identifying ex vivo fluorescence images thereof. As can be seen from the results, the photosensitizer-fucoidan conjugate remained for a long time in respective tissues in the body, including the tumor, as compared with a case where the photosensitizer is administered, and the amount of photosensitizer delivered to the tumor was also remarkably increased as compared with the free photosensitizer (free Ce6). Thus, it can be seen that the photosensitizer-fucoidan conjugate is a suitable target therapeutic agent which can exhibit an increased photodynamic therapeutic effect. FIG. 19D illustrates results obtained by freezing the obtained tumor tissues to prepare frozen sections, and then identifying, with a confocal microscope, a degree of penetration of the photosensitizer into each tumor tissue. In the control (PBS) and the free photosensitizer treatment group (free Ce6), a fluorescence signal caused by the photosensitizer was hardly observed in the tumor tissue, whereas in the photosensitizer-fucoidan conjugate treatment group, a strong fluorescence signal caused by the photosensitizer was observed in the tumor tissue. From these results, it was found that the conjugate is delivered to the tumor tissue and penetrated well into the tumor tissue, and it was found that such results accord with FIGS. 19A, 19B, and 19C. In addition, it was suggested that in a case where the photosensitizer-fucoidan conjugate is used, tumor location can be detected from the fluorescence image using optical properties thereof restored in the tumor.

### EXAMPLE 12

### Animal Test for Photodynamic Therapy-Enhancing Effect of Photosensitizer-Fucoidan Conjugate

A photodynamic therapeutic effect caused by a photosensitizer-fucoidan conjugate was evaluated in a tumor-transplanted animal model. A tumor model into which HT1080 cancer cells are subcutaneously xenografted was injected intravenously with a free photosensitizer (free Ce6) or Ce6-Fucoidan, and light irradiation (PDT) was performed on the tumor site using a 670 nm laser. To evaluate an antitumor effect, the tumor size was measured daily for 10 days, and differences between the respective groups were analyzed. For the control experimental animals, phosphate buffer containing no photosensitizer was injected intravenously.

1) Construction of Tumor Model and Analysis of Tumor Growth Inhibitory Effect

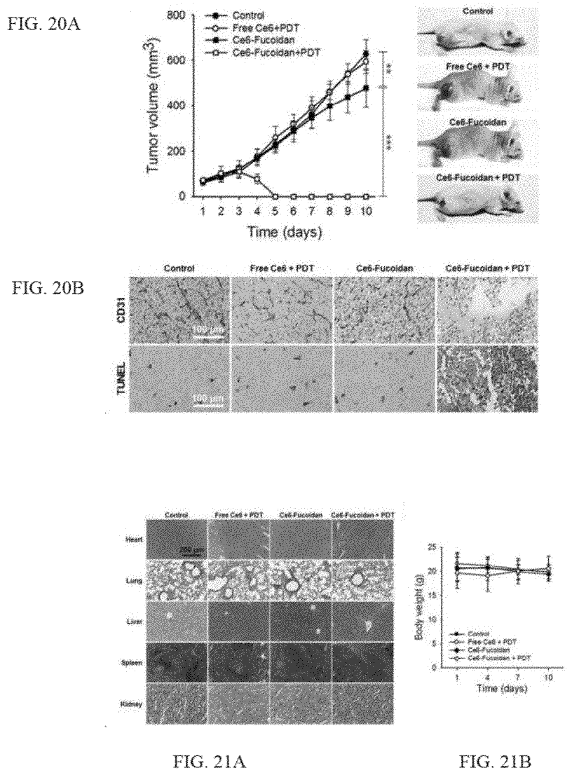

HT1080, a human fibrosarcoma cell line, was obtained from the American Type Culture Collection (ATCC, USA). The HT1080 cells were cultured, under conditions of 37° C., 5% carbon dioxide, and standard humidity, in Eagle's Minimum Essential Media (MEM) medium supplemented with 10% fetal bovine serum (FBS) and 1% penicillin/streptomycin. Nude mice (Balb/c nude) were subcutaneously injected with HT1080 cancer cells as much as 5×10⁶ cells/100 μL, and after 5 to 7 days, it was checked whether subcutaneous tumors are produced. When the tumors reached about 70 to 80 mm³ in size, the mice were divided into four groups (negative control, free Ce6+PDT, Ce6-Fucoidan, and Ce6-Fucoidan+PDT) and each experiment was performed. On day 1, free Ce6 or Ce6-Fucoidan was administered systemically through the mouse tail vein at a dose of 5 mg Ce6 equivalent/kg body weight, and the negative control was intravenously administered phosphate buffer (PBS). For the PDT group, photodynamic therapy (PDT) was performed by local laser irradiation to the tumor site on Day 2. In the PDT, a 670 nm wavelength laser was used to irradiate light at a condition of 50 mW/cm² and 20 J/cm². The tumor size was measured until Day 10 to prepare a tumor growth graph, and the tumor size was compared between the respective groups. As can be seen from the results of FIG. 20A, it was found that the best antitumor effect is observed in the Ce6-Fucoidan+PDT group, and, a tumor growth inhibitory effect was identified from the fact that on Day 10, the tumor size in the Ce6-Fucoidan group or the Ce6-Fucoidan+PDT group was 76.0% (P<0.01) or 0% (P<0.001), respectively, relative to the negative control. It was identified that the free Ce6+PDT group has no statistically significant tumor growth inhibitory effect as compared with the control. In particular, in a case where the photosensitizer-fucoidan conjugate is administered, it was possible to obtain a statistically significant anticancer effect even without light irradiation (Ce6-Fucoidan), and it was possible to obtain a very good anticancer effect with light irradiation (Ce6-Fucoidan+PDT).

2) Observation of Neovascularization Inhibitory Effect in Tumor Tissue through Tissue Staining

In order to identify neovascular distribution in tumor tissues, the mice were euthanized on Day 3 of the experiment, tumor tissues were obtained, and paraffin blocks and tissue slides were prepared. Then, the tissue slides were used to perform CD31 staining which makes it possible to identify vascular distribution. The CD31 staining was carried out as follows. Reaction was allowed to proceed using an anti-CD31 antibody (abcam) at room temperature for 2 hours, and reaction was allowed to proceed using a secondary antibody (anti-rabbit IgG-HRP) at room temperature for 1 hour. Then, color development was performed with diaminobenzidine (DAB) chromogen substrate (DAKO, Carpinteria, Calif.), counterstain was performed with Meyer's hematoxylin, and dehydration with ethanol was performed. Then, mounting was performed. Images for the stained tissue slide samples were taken with a tissue microscope. As a result, as illustrated in FIG. 20B, it was found that a similar expression level of CD31 is observed in the negative control, the free Ce6+PDT treatment group, and the Ce6-Fucoidan treatment group, whereas the Ce6-Fucoidan+PDT treatment group exhibits clearly decreased CD31 staining as compared with the other three groups. Similar to the results of FIGS. 9A, 9B, and 9C of Example 4, these results suggest a possibility that fucoidan was bound to VEGF and inhibited the VEGF signaling system, thereby inhibiting neovascularization, or that most new intratumor blood vessels were destroyed by a photodynamic therapeutic effect.

3) Observation, through Tissue Staining, of Changes in Apoptotic Induction in Tumor Tissue

In order to identify changes in cell death and apoptosis in tumor tissues, the mice were euthanized on Day 3 of the experiment for antitumor effects, tumor tissues were obtained, and paraffin blocks and tissue slides were prepared. The tissue slides were used to perform terminal deoxynucleotidyl transferase dUTP nick end labeling (TUNEL) staining. Images for the TUNEL stained tissue slide samples were taken with a tissue microscope. As a result, as illustrated in FIG. 20B, it was identified that the most apoptosis occurs in the Ce6-Fucoidan+PDT treatment group, as compared with the other groups.

### EXAMPLE 13

### In vivo Safety Evaluation of Photosensitizer-Fucoidan Conjugate

In order to evaluate in vivo safety of a photosensitizer-fucoidan conjugate, an experiment that identifies histological changes in each organ was performed. Simultaneously with performing the experiment of Example 12 to identify a tumor growth inhibitory effect, the tumor size as well as the body weight was measured until Day 10. The mice were euthanized on Day 10. Then, the heart, lung, liver, spleen, and kidney of the mice were collected, and paraffin blocks and tissue slides were prepared to perform H&E staining

Referring to FIG. 21A, it can be seen that no significant histological changes occur in all treatment groups as compared with the control. In addition, referring to FIG. 21B, it can be seen that the photosensitizer-fucoidan conjugate treatment group (Ce6-Fucoidan) or the photosensitizer-fucoidan conjugate and photodynamic therapy-combined treatment group (Ce6-Fucoidan+PDT) does not exhibit a clear decrease in body weight for 10 days as compared with the control. These results demonstrated that the photosensitizer-fucoidan conjugate is biocompatible and safe.

### EXAMPLE 14

### Analysis of Therapeutic Performance of Photosensitizer-Fucoidan Conjugate against Coronary Smooth Muscle Cells

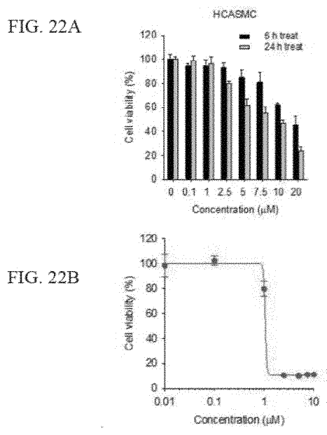

Stents are used to widen blood vessel sites narrowed due to atherosclerosis. However, proliferation of smooth muscle cells at these sites causes vascular restenosis, which is problematic. Thus, development of anticancer agent-loaded degradable stents is underway. Therefore, it was evaluated, through a cell experiment, whether the photosensitizer-fucoidan conjugate has a therapeutic effect on smooth muscle cells. FIG. 22A illustrates results obtained by subjecting human primary coronary artery smooth muscle cells (HCASMCs) to treatment with a photosensitizer-fucoidan conjugate (Ce6-Fucoidan) and a free photosensitizer (free Ce6) at various concentrations, and analyzing cell viability. It was identified that as the concentration of the photosensitizer-fucoidan conjugate increases, cell viability decreases, which seems to be due to a therapeutic effect of fucoidan which is within the conjugate.

FIG. 22B illustrates cell survival in a case where the human primary coronary artery smooth muscle cells (HCASMCs) are subjected simultaneously to treatment with the photosensitizer-fucoidan conjugate and photodynamic therapy. Light irradiation was performed using a laser of wavelength 670 nm and the light has power density of 10 J/cm². The HCASMC cells were subjected for 6 hours to treatment with Ce6, a control, and the photosensitizer-fucoidan conjugate according to the present invention at various concentrations. Then, the medium was replaced with a fresh medium, and a 670 nm laser was used to perform photodynamic therapy at 10 J/cm². The treated cells were further incubated for 24 hours in a CO₂ incubator, and then cell viability was analyzed by CCK-8 assay. As illustrated, it can be seen that the photosensitizer-fucoidan conjugate exhibits much better phototoxicity against the HCASMC cells than the conventional Ce6, and it was analyzed that the concentration (IC₅₀) value of substance required to kill about half of the cells is 1.05 μM.

From the above description, those skilled in the art will be able to understand that the present invention may be implemented in other specific modes without changing a technical spirit or an essential feature thereof. In this regard, it should be understood that the above-described examples are illustrative in all respects and not restrictive. Regarding a scope of the present invention, it should be construed that all of changed or modified forms derived from meaning and scope of the claims as described later and an equivalent concept thereto, rather than the above detailed description, are included in the scope of the present invention.

1. A conjugate, comprising a fluorescent dye or a photosensitizer covalently bonded to fucoidan.

2. The conjugate of claim 1, wherein the conjugate is formed by covalently bonding a carboxyl group of the fucoidan and an amine group of the fluorescent dye using a coupling agent.

3. The conjugate of claim 1, wherein:
the fluorescent dye is covalently bonded to fucoidan; and
the fluorescent dye is a fluorescent dye selected from the group consisting of cyanine, rhodamine, coumarin, EvoBlue, oxazine, BODIPY, carbopyronine, naphthalene, biphenyl, anthracene, phenanthrene, pyrene, carbazole, and derivatives thereof.

4. The conjugate of claim 1, wherein conjugate is formed by binding the photosensitizer and the fucoidan using a linker comprising a disulfide or diselenide bond which acts on a carboxyl group of the fucoidan.

5. The conjugate of claim 1, wherein:
the photosensitizer is covalently bonded to fucoidan; and
the photosensitizer is selected from the group consisting of:
a porphyrin-based compound selected from the group consisting of a hematoporphyrin, a porphycene, a pheophorbide, a purpurin, a chlorin, a protoporphyrin, and a phthalocyanine; and
a non-porphyrin-based compound selected from the group consisting of hypericin, rhodamine, ATTO, Rose bengal, psoralen, a phenothiazinium-based dye, and merocyanine.

6. The conjugate of claim 4, wherein the linker is at least one selected from the group consisting of selenocystamine, diselenodipropionic acid, selenocystine, cystine, and cystamine.

7. A method of conducting fluorescence imaging diagnosis of lesions for cancer or vascular diseases, comprising administering the conjugate of claim 1 to a subject in need thereof.

8. The method of claim 7, comprising detecting at least one of metastatic cancer cells overexpressing P-selectin, atherosclerotic plaques, neovascular endothelial cells, and platelet-rich thrombi.

9. A method of conducting photodynamic therapy (PDT) to prevent or treat cancer, comprising administering the conjugate of claim 1 to a subject in need thereof.

10. The method of claim 9, further comprising irradiating a prevention or treatment site with light.

11. The method of claim 9, wherein the conjugate is delivered cancer-selectively.

12. The method of claim 9, wherein the conjugate inhibits neovascularization by binding to vascular endothelial growth factor (VEGF).

13. A method for preventing or treating vascular restenosis, comprising administering the conjugate of claim 1 to a subject in need thereof.
